(19) United States
(12) Patent Application Publication
(10) Pub. No.: US 2016/0109642 A1
(43) Pub. Date: Apr. 21, 2016

(54) LIGHTING DEVICE FOR A MAGNETIC RESONANCE IMAGING SYSTEM APERTURE

(71) Applicant: Siemens Healthcare GmbH, Erlangen (DE)

(72) Inventors: Jian Hua Pei, Shenzhen (CN); Wen Qiang You, Shenzhen (CN); Xue Feng Zhao, Shenzhen (CN)

(73) Assignee: Siemens Healthcare GmbH, Erlangen (DE)

(21) Appl. No.: 14/884,812

(22) Filed: Oct. 16, 2015

(30) Foreign Application Priority Data
Oct. 16, 2014 (CN) 201420600402.0

(51) Int. Cl. F21V 8/00; A61B 5/055

(52) U.S. Cl. CPC G02B 6/0055; G02B 6/0073; G02B 6/0051; G02B 6/0091; A61B 5/055

(57) ABSTRACT

A lighting device for an MRI system aperture has at least one LED lamp and a light guide plate, the light guide plate having an interior surface, an exterior surface and multiple end faces. The end faces include a first end face, a second end face and one or more other end faces. Light rays from the LED lamp enter the light guide plate through the first end face, and exit the light guide plate through the exterior surface. The inner surface is opposite the outer surface, and the second end face is opposite the first end face. This lighting device for an MRI system aperture reduces the volume occupied in the MRI system aperture and at the same time the light rays are gentle and produce an effect of gradual change, protecting the examination subject's eyes and dispelling nervous feelings and claustrophobia in the examination subject.

### BACKGROUND OF THE INVENTION

1. Field of the Invention

The present invention concerns magnetic resonance imaging, in particular an aperture lighting device for a magnetic resonance imaging scanner.

2. Description of the Prior Art

Magnetic resonance imaging (MRI) is a modality in which the phenomenon of magnetic resonance is utilized for the purpose of imaging. The basic principles of magnetic resonance are as follows. When an atomic nucleus contains a single proton, as is the case for nuclei of hydrogen atoms that are present throughout the human body, this proton exhibits spin motion and resembles a small magnet (dipole). The spin axes of these small magnets lack a definite pattern, so when an external magnetic field is applied, the small magnets will be rearranged according to the magnetic force lines of the external field - specifically, they will align in two directions, either parallel or anti-parallel to the magnetic force lines of the external magnetic field. The direction parallel to the magnetic force lines of the external magnetic field is called the positive longitudinal axis, while the direction anti-parallel to the magnetic force lines of the external magnetic field is called the negative longitudinal axis. The atomic nuclei now only have a longitudinal magnetization component, which has both a direction and a magnitude. A radio frequency (RF) pulse of a specific frequency is radiated to excite the atomic nuclei in the external magnetic field such that their spin axes deviate from the positive longitudinal axis or negative longitudinal axis, giving rise to resonance—this is the phenomenon of magnetic resonance. Once the spin axes of the excited atomic nuclei have deviated from the positive or negative longitudinal axis, the atomic nuclei have a transverse magnetization component.

Once emission of the RF pulse has ended, the excited atomic nucleus emits an echo signal, gradually releasing the absorbed energy in the form of electromagnetic waves, such that its phase and energy both return to the pre-excitation state. An image can be reconstructed by subjecting the echo signal emitted by atomic nuclei to further processing, such as spatial encoding.

During scanning with an MRI system in the prior art, a patient may feel nervous or even claustrophobic due to the small size of the MRI system aperture and the presence of a strong magnetic field therein. Therefore the issue of whether an MRI system can provide a comfortable environment for patients during scanning is very important. A good aperture lighting design helps patients to feel more comfortable.

Furthermore, during scanning with an MRI system in the prior art, an increase in the temperature of gradient coils in the MRI system may be transferred to the patient's body, making the patient feel hot. The doctor must observe the state of the patient's face, and on this basis stop the scan if necessary. Aperture lighting must provide the doctor with the necessary light levels, to ensure that the doctor can distinguish the patient's facial states.

FIG. 5 is a schematic diagram of an aperture lighting device of an MRI system in the prior art. As FIG. 5 shows, a light source 500 of the aperture lighting of the MRI system in the prior art is installed at an end (rear side) of the MRI system aperture, and is generally a light-emitting diode (LED). The patient can easily look directly at the light source and thereby feel uncomfortable, and may even suffer damage to the eyesight.

Siemens Invention with publication number CN 203524689 U describes in detail an aperture structure of an MRI system as well as a lighting device for an MRI system aperture.

### SUMMARY OF THE INVENTION

In view of the above, the present invention provides a lighting device for an MRI system aperture, having one or more LED lamps and a light guide plate. The light guide plate has an interior surface, an exterior surface and multiple end faces, wherein the end faces include a first end face, a second end face and one or more other end faces. Light rays from the LED lamp enter the light guide plate through the first end face, light rays from the LED lamp exit the light guide plate through the exterior surface. The interior surface is opposite the exterior surface, and the second end face is opposite the first end face.

Preferably, a first reflector is disposed at the second end face.

Preferably, one or more second reflectors are disposed at the other end face or faces.

Preferably, a third reflector is disposed at the interior surface.

Preferably, a light scattering film is located on the exterior surface.

Preferably, a transparent cover is situated on the exterior surface.

Preferably, a housing, in which the LED lamp and the light guide plate are disposed, has a power supply connected electrically to the LED lamp.

Preferably, a first fixing portion is disposed at the interior surface or the end face, and the housing also has a second fixing portion that fits the first fixing portion.

The present invention also provides an MRI system having one or more lighting devices according to any of the above embodiments.

Preferably, the lighting device(s) is/are located on two sides or below a patient table of the MRI system.

Preferably, the lighting device(s) is/are disposed on an inner wall of the MRI system aperture along an axial direction.

Preferably, the first end face is an end face of the lighting device along the axial direction of the aperture.

Preferably, the system has at least one slot in each of which an aperture lighting device is inserted.

In summary, according to a particular embodiment of the present invention, the volume occupied in the MRI system aperture is reduced as far as possible and at the same time, the aperture lighting device for an MRI system according to the present invention is provided, with an LED lamp at only one end face of the light guide plate, so that the light rays are gentle and produce an effect of gradual change, protecting the examination subject's eyes and dispelling nervous feelings and claustrophobia in the examination subject.

### BRIEF DESCRIPTION OF THE DRAWINGS

FIG. 1 schematically illustrates a lighting device for an MRI system aperture according to an embodiment of the invention.

FIG. 2 schematically illustrates an MRI system aperture according to an embodiment of the invention.

FIG. 3 schematically illustrates an aperture, and a lighting device for the aperture, of an MRI system according to an embodiment of the present invention.

FIG. 4 schematically illustrates a housing of a lighting device for an MRI system aperture according to an embodiment of the present invention.

FIG. 5 schematically illustrates a lighting device for an MRI system aperture in the prior art.

### DESCRIPTION OF THE PREFERRED EMBODIMENTS

The present invention is explained in further detail below by means of particular embodiments, to clarify the object, technical solution and advantages thereof.

The core of the technical solution of the lighting device for an MRI system aperture according to a particular embodiment of the present invention is as follows: in order to provide a comfortable scanning environment for a patient, the lighting device for an MRI system aperture according to a particular embodiment of the present invention is installed on an inner wall of the MRI system aperture and a light guide plate is used to change the direction of light rays from an LED lamp, thereby reducing as far as possible the volume occupied in the MRI system aperture; at the same time, the aperture lighting device for an MRI system according to a particular embodiment of the present invention is provided with an LED lamp at only one end face of the light guide plate, so that the light rays are gentle and produce an effect of gradual change, protecting the examination subject's eyes and dispelling nervous feelings and claustrophobia in the examination subject.

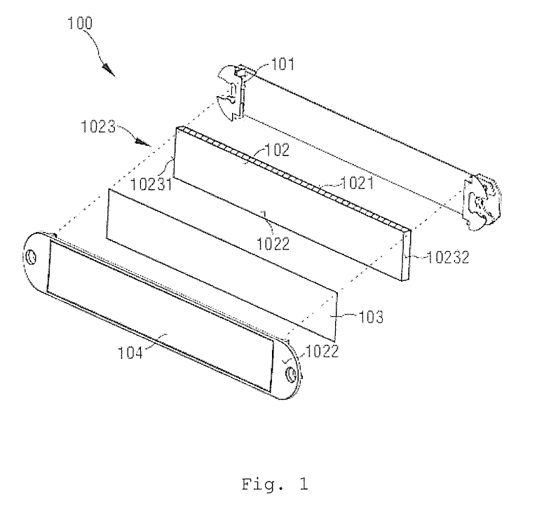

FIG. 1 is a schematic diagram of a lighting device for an MRI system aperture according to a particular embodiment of the present invention. As FIG. 1 shows, the lighting device 100 for an MRI system aperture according to a particular embodiment of the present invention has one or more LED lamp(s) 101 and a light guide plate 102, the light guide plate 102 comprising an inner surface 1021, an outer surface 1022 and multiple end faces 1023, wherein the end faces comprise a first end face 10231, a second end face 10232 and one or more other end face(s), light rays of the LED lamp 101 exit the light guide plate 102 through the outer surface 1022, the inner surface 1021 is opposite the outer surface 1022, and light rays of the LED lamp 101 enter the light guide plate 102 through the first end face 10231.

As FIG. 1 shows, the first end face 10231 is the left end face (or lower end face) of the light guide plate 102, and the second end face 10232 (i.e. the right end face (or upper end face) of the LED lamp) is wholly or partly opposite the first end face 10231. When the light guide plate 102 is used, long-distance lighting can be achieved by using only a small number of LED lamps 101, so that costs are saved.

The lighting device 100 for an MRI system aperture according to a particular embodiment of the present invention as shown in FIG. 1 is columnar, with both the inner surface 1021 and the outer surface 1022 being rectangular. However, a lighting device for an MRI system aperture according to another particular embodiment of the present invention may have another shape, such as a pentagon, a hexagon, another polygon or a circle or ellipse, so as to suit a variety of different installation environments and produce a variety of different visual effects.

The lighting device 100 for an MRI system aperture according to a particular embodiment of the present invention as shown in FIG. 1 also has a first reflector disposed at the second end face 10232. Since the second end face 10232 is wholly or partly opposite the first end face 10231, the first reflector can reflect light rays emitted from the LED lamp 101 disposed at the first end face 10231 and thereby increase the light ray utilization rate.

Moreover, the lighting device 100 for an MRI system aperture according to a particular embodiment of the present invention as shown in FIG. 1 also has a second reflector disposed on the other end face, so that the second reflector can reflect light rays emitted from the LED lamp 101 disposed at the first end face 10231 and thereby increase the light ray utilization rate.

Moreover, the lighting device 100 for an MRI system aperture according to a particular embodiment of the present invention as shown in FIG. 1 also has a third reflector disposed at the inner surface, the inner surface facing the aperture of the MRI system, so that the third reflector can reflect light rays emitted from the LED lamp 101 disposed at the first end face 10231 and thereby increase the light ray utilization rate.

Moreover, the lighting device 100 for an MRI system aperture according to a particular embodiment of the present invention as shown in FIG. 1 also has a light scattering film 103 located on the outer surface 1022. The function of the light scattering film 103 is to scatter light rays so as to obtain a homogeneous and gentle light ray effect, and thereby create a comfortable light environment while avoiding damage to the eyes of medical personnel and the patient.

Moreover, the lighting device 100 for an MRI system aperture according to a particular embodiment of the present invention as shown in FIG. 1 also has a transparent cover 104 located on the outer surface 1022. The function of the transparent cover 104 is to protect components underneath the transparent cover 104 while scattering light rays further so as to obtain a homogeneous and gentle light ray effect.

Moreover, the LED lamp 101, light guide plate 102, light scattering film 103 or transparent cover 104 of the lighting device 100 for an MRI system aperture according to a particular embodiment of the present invention as shown in FIG. 1 are fixed in place by adhesion, snap fastening or locking (bolt and nut). More specifically, the light guide plate 102 is encapsulated by means of reflectors or reflecting films disposed on the first end face 10231, second end face 10232 and other end face(s), to avoid light leakage.

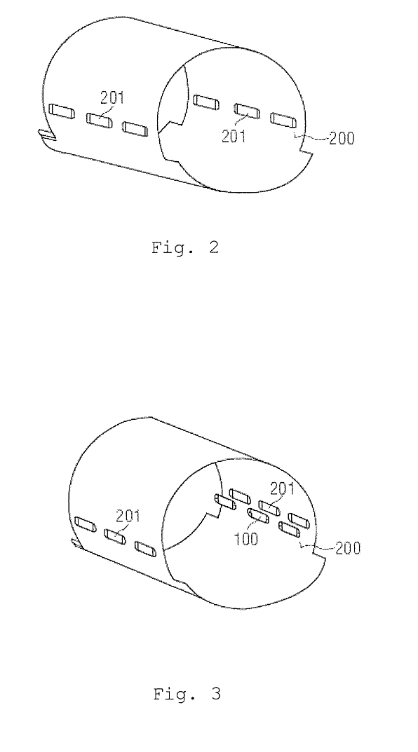

FIG. 2 is a schematic diagram of an MRI system aperture according to a particular embodiment of the present invention. FIG. 3 is a schematic diagram of an aperture, and a lighting device for the aperture, of an MRI system according to a particular embodiment of the present invention. As shown in FIGS. 2 and 3, the lighting device 100 for an MRI system aperture according to a particular embodiment of the present invention is in the aperture 200 of the MRI system according to a particular embodiment of the present invention; aperture lighting devices 100 are disposed on an inner wall of the MRI system aperture along an axial direction, wherein the aperture 200 comprises multiple slots 201, with the inner diameter of each slot 201 matching the periphery of the aperture lighting device 100, so that multiple aperture lighting devices 100 can be inserted individually into multiple slots 201. Although slots 201 are provided in the inner wall of the aperture 200 of the MRI system according to a particular embodiment of the present invention, these slots have only a very small effect on the rigidity of the aperture 200; moreover, the space occupied by the lighting device inside the aperture is saved, so that the examination subject is provided with more space, and is therefore more comfortable. The lighting devices 100 are located on two sides or below a patient table of the MRI system; this design can prevent light rays from shining directly at the examination subject and thereby protect the examination subject from harm.

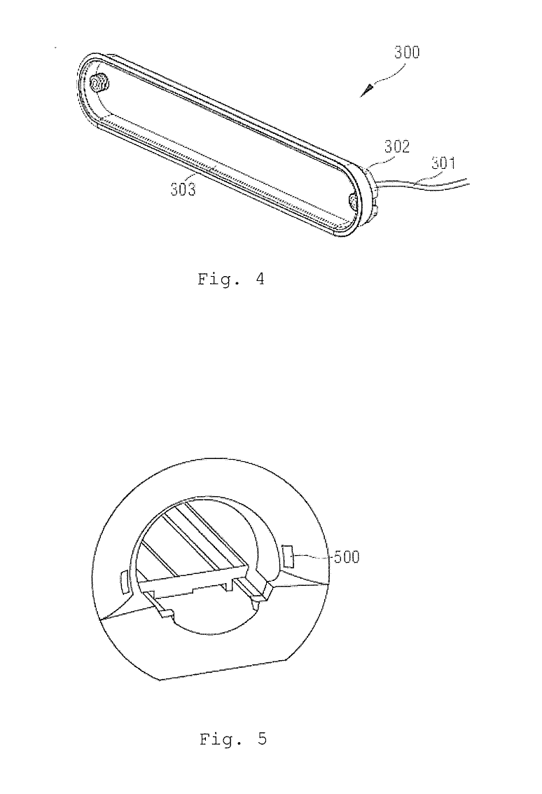

Furthermore, FIG. 4 is a schematic diagram of a housing of a lighting device for an MRI system aperture according to a particular embodiment of the present invention. As FIG. 4 shows, the housing 300 of a lighting device for an MRI system aperture according to a particular embodiment of the present invention has an electric wire 301, a power supply interface 302 and a bottom cover 303. The lighting device 100 also has a first fixing portion disposed at the inner surface or the end face, and the housing 300 also has a second fixing portion which fits the first fixing portion. The housing 300 is inserted in the slot 201, and will not be removed during maintenance, therefore the lighting device for an MRI system aperture according to a particular embodiment of the present invention can provide friendly and flexible maintenance.

Although modifications and changes may be suggested by those skilled in the art, it is the intention of the inventors to embody within the patent warranted hereon all changes and modifications as reasonably and properly come within the scope of their contribution to the art.

We claim as our invention:

1. A lighting device for a magnetic resonance imaging (MRI) system aperture, comprising:
one LED lamp and a light guide plate;
said light guide plate comprising an interior surface, an exterior surface and multiple end faces, and the end faces including a first end face, a second end face and at least one other end face opposite the exterior surface, and the second end face opposite the first end face; and
said LED lamp and said light guide plate being oriented relative to each other to cause light rays from the LED lamp to enter the light guide plate through the first end face, and light rays from the LED lamp exit the light guide plate through the outer surface, the inner surface.

2. The lighting device as claimed in claim 1, further comprising a first reflector disposed at the second end face.

3. The lighting device as claimed in claim 2, further comprising at least one second reflector disposed at each other end face.

4. The lighting device as claimed in claim 2, further comprising a third reflector disposed at the interior surface.

5. The lighting device as claimed in claim 1, further comprising a light scattering film located on the exterior surface; and/or a transparent cover located on the exterior surface.

6. The lighting device as claimed in claim 1, further comprising a housing in which the LED lamp and the light guide plate are disposed, the housing comprising a power supply connected electrically to the LED lamp.

7. The lighting device as claimed in claim 6, further comprising a first fixing portion disposed at the interior surface or the end face, and wherein the housing comprises a second fixing portion that fits the first fixing portion.

8. A magnetic resonance imaging (MRI) system comprising:
an MRI scanner having a patient-receiving aperture therein;
at least one lighting device mounted in said aperture; and
said at least one lighting device comprising one LED lamp and a light guide plate, said light guide plate comprising an interior surface, an exterior surface and multiple end faces, and the end faces including a first end face, a second end face and at least one other end face opposite the exterior surface, and the second end face opposite the first end face, and said LED lamp and said light guide plate being oriented relative to each other to cause light rays from the LED lamp to enter the light guide plate through the first end face, and light rays from the LED lamp exit the light guide plate through the outer surface, the inner surface.

9. The MRI system as claimed in claim 8 comprising a patient table that is movable through said aperture, and wherein said at least one lighting device is mounted in said aperture at a location selected from the group consisting of a location level with a side of said patient table, and a location below said patient table.

10. The MRI system as claimed in claim 8, the lighting device is disposed on an inner wall of the MRI system aperture along an axial direction.

11. The MRI system as claimed in claim 10, wherein the first end face is an end face of the lighting device along an axial direction of the aperture.

12. The MRI system as claimed in claim 8, comprising at least one slot in which the aperture lighting device is inserted.
